(19) United States
(12) Patent Application Publication
(10) Pub. No.: US 2024/0009260 A1
(43) Pub. Date: Jan. 11, 2024

(54) COMPOSITION FOR PREVENTING OR TREATING COLITIS, COMPRISING PYRUS USSURIENSIS EXTRACT OR PYRUS USSURIENSIS FERMENTATION PRODUCT AS ACTIVE INGREDIENT

(71) Applicant: NAM KOOK KIM, GANGWON-DO (KR)

(72) Inventor: NAM KOOK KIM, GANGWON-DO (KR)

(21) Appl. No.: 18/037,079

(22) PCT Filed: Dec. 15, 2021
(86) PCT No.: PCT/KR2021/019032

§ 371 Date: May 16, 2023

(30) Foreign Application Priority Data
Dec. 16, 2020 (KR) 10-2020-0176817

(51) Int. Cl. A61K 36/73; A61P 1/00

(52) U.S. Cl. CPC A61K 36/73; A61P 1/00; A61K 2236/19; A61K 2236/333

(57) ABSTRACT

The present disclosure relates to a pharmaceutical composition, a food composition, and a health functional food composition for prevention or treatment of colitis including an extract of pyrus ussuriensis or a fermented product of pyrus ussuriensis as an active ingredient, and a treatment method for colitis including administering the pharmaceutical composition to a subject suspected of colitis. The extract of pyrus ussuriensis or the fermented product of pyrus ussuriensis provided by the present disclosure has anti-inflammatory activity that significantly reduces the main symptoms of colitis, while suppressing inflammatory cytokines, thereby it can be widely used in the prevention or treatment of colitis.

### CROSS-REFERENCE TO RELATED PATENT APPLICATION

This application is a U.S. national stage application claiming the benefit of International Application No. PCT/KR2021/019032, filed on Dec. 15, 2021, the entire content of which is incorporated herein by reference in its entirety.

### FIELD OF THE DISCLOSURE

The present disclosure relates to a pharmaceutical composition, a food composition, and a health functional food composition for prevention or treatment of colitis including an extract of pyrus ussuriensis or a fermented product of pyrus ussuriensis as an active ingredient, and relates to a treatment method for colitis including administering the pharmaceutical composition to a subject other than humans.

### BACKGROUND OF THE DISCLOSURE

Pyrus ussuriensis is a tree that grows naturally nationwide, and blooms in April-May, and the pyrus ussuriensis fruit matures in September-October. The pyrus ussuriensis is a round yellow fruit with a diameter of 3-4 cm, and has a very high acidity due to its sour taste, so it is usually processed and consumed rather than consumed immediately as a fruit. In Korea, the fruit of the pyrus ussuriensis has been used for medicinal purposes such as cough, diarrhea, and thirst (Encyclopedia of Korean Medicine), and has been used as medicines with the same efficacy as pears as folk remedies, but they are known to have better efficacy than pear trees. The pyrus ussuriensis is mainly used for medicinal purposes to relieve cough sputum, fever, tuberculosis, constipation, etc. (Herbal Encyclopedia), and the antioxidant efficacy of the pyrus ussuriensis has been reported (Food Chemistry 152 (2014) 531-538), but the efficacy of the pyrus ussuriensis in treating colitis has not been studied at all.

On the other hand, colitis is a type of inflammatory bowel disease occurring in the colon and is one of the chronic diseases of modern people that causes bleeding accompanied by multiple ulcers. Although the cause of colitis has not yet been clearly identified, it is known to be related to genetic factors, bacterial infections, environmental factors such as excessive drinking and irregular eating habits, excessive immune response in the body against intestinal microorganisms, and obesity, etc. Symptoms of colitis include loose stools or diarrhea with blood and mucus, severe abdominal pain, dehydration due to diarrhea, anemia, weight loss, general fatigue, and fever, etc. As the elderly population increases worldwide, the number of colorectal cancer patients is also increasing along with the increase in intractable chronic colitis patients.

Existing medicines used to treat colitis include steroidal immunosuppressants, 5-aminosalicylic acid (5-ASA) medicines that block the generation of prostaglandin (e.g., sulfasalazine), and mesalazine, etc. However, the use of these therapeutic agents is limited because they have a negligible treatment effect on colitis and cause serious side effects such as abdominal fullness, headache, rash, liver disease, leukopenia, agranulocytosis, and male infertility, etc. Therefore, there is a demand for the development of a colitis therapeutic agent that is effective, safe, and does not cause side effects.

Under this background, the present inventors have made intensive research efforts to develop a method for improving and treating colitis using natural products that are more stable than synthetic medicines, and completed the present invention by confirming that the extract of pyrus ussuriensis and the fermented product of pyrus ussuriensis have an effect of treating colitis.

The present disclosure was developed with the support of Biomedical Hub joint research and development project.

### SUMMARY OF THE DISCLOSURE

One purpose of the present disclosure is to provide a pharmaceutical composition for prevention or treatment of colitis including an extract of pyrus ussuriensis or a fermented product of pyrus ussuriensis as an active ingredient.

Another purpose of the present disclosure is to provide a treatment method for colitis including administering the pharmaceutical composition to a subject other than humans.

Another purpose of the present disclosure is to provide a food composition for prevention or improvement of colitis including an extract of pyrus ussuriensis or a fermented product of pyrus ussuriensis as an active ingredient.

Another purpose of the present disclosure is to provide a health functional food composition for prevention or improvement of colitis including an extract of pyrus ussuriensis or a fermented product of pyrus ussuriensis as an active ingredient.

### TECHNICAL SOLUTION

A detailed description thereof is as follows. Each description and embodiment disclosed in the present disclosure may also be applied to each other description and embodiment. That is, all combinations of the various elements disclosed in the present disclosure belong to the categories of the present disclosure. Further, it is not to be considered that the categories of the present disclosure are limited by the specific descriptions described below.

In addition, those skilled in the art will recognize or confirm various equivalents with respect to a specific aspect of the present disclosure described herein using only ordinary experiments. Also, these equivalents are intended to be included in the present disclosure.

In order to achieve the above purposes, the present disclosure provides a pharmaceutical composition for prevention or treatment of colitis including an extract of pyrus ussuriensis or a fermented product of pyrus ussuriensis as an active ingredient.

The “pyrus ussuriensis” of the present disclosure is a fruit of Pyrus ussuriensis classified as Pyrus of the Rosaceae family, and is distributed throughout Korea, Russia, Japan and Manchuria, China, etc. The pyrus ussuriensis is a round yellow fruit with a diameter of 3-4 cm. Although the antioxidant efficacy of the pyrus ussuriensis has been reported, the efficacy of treating colitis has not been studied, and in the present disclosure, it was first identified that the extract of pyrus ussuriensis or the fermented product of pyrus ussuriensis has a therapeutic effect on colitis.

The pyrus ussuriensis of the present disclosure may be raw, dried, or processed, and commercially available ones may be purchased and used, or those harvested or cultivated in nature may be used, but is not limited thereto.

The term “extract” of the present disclosure refers to a product such as a liquid component obtained by immersing a desired material in various solvents and then extracting the desired material at room temperature or a warm state for a certain period of time, or a solid substance obtained by removing the solvent from the above liquid component. In addition, it can be comprehensively interpreted as including all diluent of the above output, concentrates thereof, adjusted products thereof, and purified products thereof, etc. Accordingly, the extract of pyrus ussuriensis provided by the present disclosure may be interpreted as including an extract obtained by extracting the pyrus ussuriensis, a purified product obtained by enzymatic decomposition of the above extract, or a mixture thereof, the extract itself and extracts of all formulations that can be formed using the extract.

The method of extracting the pyrus ussuriensis of the present disclosure is not specifically limited, and the pyrus ussuriensis can be extracted according to a method commonly used in the art. Non-limiting examples of the extraction method may include a hot water extraction, an ultrasonic extraction, a filtration, a reflux extraction, etc., which may be performed alone or in combination of two or more methods.

In the present disclosure, the type of solvent used for the extraction is not specifically limited, and any solvent known in the art may be used. Non-limiting examples of such extraction solvent may include water, alcohol, or a mixture solvent thereof, which may be used alone or in combination of two or more types. In the case of using alcohol as a solvent, specifically, alcohol having 1 to 4 carbon atoms can be used.

The term “fermented product” of the present disclosure may be obtained by fermenting the pyrus ussuriensis, and specifically may be obtained by mixing and reacting the pyrus ussuriensis and sugar. The sugar may be selected from, sucrose, maltose, glucose, fructose, and sugar, but is not limited thereto. In the present disclosure, “fermented product” refers to a product of liquid component obtained through the fermentation process, and refers to a solid substance obtained by removing the solvent from the above liquid component.

The fermented product of the present disclosure may be a mixture of pyrus ussuriensis and sugar in a weight ratio of 3:7 to 9:1, a weight ratio of 4:6 to 8:2, or a weight ratio of 5:5 to 7:3, but is not limited thereto.

The “colitis” of the present disclosure is a disease in which inflammation occurs in the colon due to various causes, and is largely classified into infectious colitis and non-infectious colitis according to the cause. Infectious colitis includes viral enteritis (norovirus, rotavirus), bacterial enteritis (cholera, E. coli, dysentery, typhoid fever, yersinia, Campylobacter), protozoal colitis (amoeba), and pseudomembranous colitis depending on the type of infectious bacteria. Non-infectious colitis includes inflammatory bowel disease, radiation-induced colitis, ischemic colitis, Behcet's colitis, and drug-induced colitis, etc.

In the present disclosure, the colitis may be at least one or more selected from the group consisting of acute colitis, bacterial colitis, viral colitis, pseudomembranous colitis, inflammatory bowel disease, chronic colitis, ulcerative colitis, Crohn's disease, ischemic colitis, Behcet's colitis, drug-induced colitis, collagenous colitis, lymphocytic colitis, and radiation colitis, but is not limited thereto.

Symptoms of colitis include loose stools or diarrhea with blood and mucus, severe abdominal pain, dehydration due to diarrhea, anemia, weight loss, general fatigue, and fever, etc.

In one specific embodiment of the present disclosure, in the case that an extract of pyrus ussuriensis or a fermented product of pyrus ussuriensis was administered to a colitis animal model, the effects of reducing disease activity index (DAI), reducing diarrhea index, and reducing bloody stool index were confirmed (Embodiment 3-1), and it was confirmed that the extract and fermented product of pyrus ussuriensis were effective in improving colitis symptoms.

The term “prevention” of the present disclosure refers to all activities that suppress or delay the occurrence of colitis using a composition including the extract of pyrus ussuriensis or the fermented product of pyrus ussuriensis as an active ingredient.

The term “treatment” of the present disclosure refers to all activities that control or alleviate the symptoms of colitis using a composition including the extract of pyrus ussuriensis or the fermented product of pyrus ussuriensis as an active ingredient.

The “pharmaceutical composition” of the present disclosure may further include a pharmaceutically acceptable carrier, excipient or diluent. Such pharmaceutically acceptable carriers, excipients, or diluents may be non-naturally occurring. Specifically, the composition may be used by being formulated in the form of oral formulations such as powders, granules, tablets, solutions, capsules, suspensions, emulsions, syrups, aerosols and the like, external preparations, suppositories and sterile injection solutions, respectively, according to conventional methods. In the present disclosure, carriers, excipients, and diluents that may be included in the pharmaceutical composition include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. In the case of formulating, it is prepared by using diluents or excipients such as commonly used fillers, extenders, binders, wetting agents, disintegrants, and surfactants, etc. Solid formulations for oral administration include tablets, pills, powders, granules, capsules and the like. These solid formulations are prepared by mixing the above extract and its fractions with at least one excipient, for example, starch, calcium carbonate, sucrose or lactose, gelatin and the like. In addition to the simple excipients, lubricants such as magnesium taurate and talc are also used. Liquid formulations for oral use may include suspensions, solutions for oral use, emulsions and syrups, and various excipients such as wetting agents, sweeteners, aromatics, and preservatives may be included besides water and liquid paraffin which are commonly used as simple diluents. Formulations for parenteral administration include sterilized aqueous solutions, non-aqueous solvents, suspensions, emulsions, freeze-dried formulations, and suppositories. Propylene glycol, polyethylene glycol, vegetable oils such as olive oil, and injectable esters such as ethyl oleate may be used as non-aqueous solvents and suspensions. As a base for the suppository, witepsol, macrogol, tween 61, cacao butter, laurin fat, glycerogelatin and the like may be used.

Another aspect of the present disclosure provides a treatment method for colitis including administering to a subject other than humans a pharmaceutical composition including the extract of pyrus ussuriensis or the fermented product of pyrus ussuriensis as an active ingredient.

The “extract of pyrus ussuriensis”, “fermented product of pyrus ussuriensis”, “pharmaceutical composition”, and “colitis” are as described above.

The term “subject” of the present disclosure may refer to all animals, including humans, who have or are likely to develop colitis. The animal may be not only humans but also mammals such as cattle, horses, sheep, pigs, goats, camels, antelopes, dogs, and cats that require treatment for similar symptoms, but is not limited thereto.

Specifically, the prevention or treatment method of the present disclosure may include administering the composition in a pharmaceutically effective amount to a subject having or at risk of developing colitis.

The term “administering” used in the present disclosure refers to introducing the pharmaceutical composition of the present disclosure to a patient by any suitable method, and the route of administering the composition of the present disclosure may be through various oral or parenteral routes as long as it can reach the target tissue.

The active ingredient of the pharmaceutical composition of the present disclosure may vary depending on the age, gender, weight, pathological condition and severity of a subject to be administered, the route of administration, or the judgment of the prescriber. Determination of application amount based on these factors is within the level of those skilled in the art, and its daily administration dose may be, for example, 0.1 mg/g/day to 100 mg/g/day, more specifically 5 mg/g/day to 50 mg/g/day. The frequency of administration of the composition of the present disclosure is not particularly limited thereto, but it is possible to be administered once a day or several times by dividing the dose. This dose is not intended to limit the scope of the present disclosure in any way.

Another aspect of the present disclosure is to provide a food composition for prevention or improvement of colitis including an extract of pyrus ussuriensis or a fermented product of pyrus ussuriensis as an active ingredient.

Another aspect of the present disclosure is to provide a health functional food composition for prevention or improvement of colitis including an extract of pyrus ussuriensis or a fermented product of pyrus ussuriensis as an active ingredient.

The “extract of pyrus ussuriensis”, “fermented product of pyrus ussuriensis”, “colitis” and “prevention” are as described above.

The term “improvement” in the present disclosure refers to all activities that improve or benefit the symptoms of suspected or affected subjects by using the composition.

The term “food” of the present disclosure includes all foods in the usual sense, such as meat, sausage, bread, chocolate, candy, snacks, confectionery, pizza, ramen, other noodles, chewing gum, dairy products including ice cream, various soups, beverages, tea, drinks, alcoholic beverages, vitamin complexes, and health functional foods and the like, and is not limited thereto as long as it may include the extract of pyrus ussuriensis or the fermented product of pyrus ussuriensis of the present disclosure. In addition, the food composition may be prepared by being added to squeeze, tea, jelly, juice and the like produced with the extract of pyrus ussuriensis or the fermented product of pyrus ussuriensis according to the present disclosure as a main component, and it may include forms such as pills, powders, granules, infusions, tablets, capsules or solutions, etc.

In the case of preparing the food composition, it may be prepared by adding raw materials and ingredients commonly added in the art, and the type is not particularly limited. For example, the food composition may include various herbal extracts, food-acceptable food auxiliary additives, or natural carbohydrates as additional components, as in conventional foods, but is not limited thereto. The mixing amount of the active ingredient may be appropriately determined depending on the purpose of use, and since the composition of the present disclosure contains an extract or a fermented product derived from a natural product as an active ingredient, there is no problem in terms of stability, so the mixing amount is not particularly limited.

The term “health functional food” of the present disclosure is the same term as a food for special health use (FoSHU), and it refers to a food made of specific ingredients or manufactured and processed by methods of extracting, concentrating, refining, and mixing specific ingredients contained in food materials for the purpose of supplementing health. The composition for health functional food refers to a food designed and processed to sufficiently exert bioregulatory functions such as biodefense, regulation of biorhythm, prevention and recovery of disease and the like with respect to a living body by the above ingredients, and the composition for health functional food may perform functions related to prevention and recovery of disease. The health functional food of the present disclosure can be used interchangeably with terms known in the art, such as functional food.

### EFFECT OF THE INVENTION

The extract of pyrus ussuriensis or the fermented product of pyrus ussuriensis provided by the present disclosure has anti-inflammatory activity that significantly reduces the main symptoms of colitis, while suppressing inflammatory cytokines, and may be widely used in the prevention or treatment of colitis.

### BRIEF DESCRIPTION OF THE DRAWINGS

FIG. 1 is a diagram illustrating a series of experimental schedules for orally administering the composition of the present disclosure to a DSS-induced colitis animal model.

FIG. 2(a) and FIG. 2(b) are diagrams illustrating a diarrhea index of a colitis animal model (FIG. 2(a) *p<0.05, ****p<0.0001 vs Con, #p<0.05 vs DSS), (FIG. 2(b) #p<0.05, ##p<0.01 vs DSS).

FIG. 3(a) and FIG. 3(b) are diagrams illustrating a bloody stool index of a colitis animal model (FIG. 3(a) **p<0.01, ****p<0.0001 vs Con, #p<0.05, ##p<0.01, ###p<0.001 vs DSS).

FIG. 4(a) and FIG. 4(b) are diagrams illustrating a DAI score of a colitis animal model (FIG. 4(a) ***p<0.001, ****p<0.0001 vs Con).

FIG. 5(a) and FIG. 5(b) are analyses of the concentration of inflammatory factors in a colitis animal model, FIG. 5a is a graph measuring the concentrations of IL-6 and IL-1β, which are inflammatory cytokines (*p<0.05, “p<0.01 vs Con, #p<0.05 vs DSS), and FIG. 5b is a graph measuring the concentration of MPO, which is an inflammatory response factor (”p<0.01 vs Con, #p<0.05, ####p<0.0001 vs DSS).

### DETAILED DESCRIPTION OF THE EXEMPLARY EMBODIMENTS

### Embodiments

Hereinafter, the present disclosure will be described in more detail through embodiments. These embodiments are intended to provide a more detailed explanation of the present disclosure, and the scope of the present disclosure is not limited thereto.

### Embodiment 1. Preparation of Extract of Pyrus Ussuriensis and Fermented Product of Pyrus Ussuriensis

### 1-1. Preparation of Hot Water Extract of Pyrus Ussuriensis

The dried pyrus ussuriensis was pulverized and used as powder. 60 g of pyrus ussuriensis powder was put in a 1 L extraction flask, 10 times distilled water was added, and then it was connected to a reflux cooling extraction device and extracted at 90° C. for 4 hours. The extract was filtered through a Watman No. 1 filter paper, and then it was frozen in a freezer at −80° C. and freeze-dried. The prepared hot water extract powder of pyrus ussuriensis was suspended in PBS and used.

### 1-2. Preparation of Ethanol Extract of Pyrus Ussuriensis

50 grams of pyrus ussuriensis powder was put in a 1 L extraction flask, 50% ethanol of 10 times was added, and then it was connected to a reflux cooling extraction device and extracted twice for 2 hours at 70° C. The extract was filtered through a Watman No. 1 filter paper, and then the ethanol was removed using a vacuum concentrator. The extract concentration was frozen at −80° C. and freeze-dried. The prepared ethanol extract powder of pyrus ussuriensis using was suspended in PBS and used.

### 1-3. Preparation of Fermented Product of Pyrus Ussuriensis

After cleaning the raw pyrus ussuriensis that was harvested on the same day more than five times, moisture was removed and dried in the shade. The pyrus ussuriensis and sugar were mixed at a ratio of 6:4, mixed in a loess vacuum jar. When the osmotic pressure phenomenon progressed, the pyrus ussuriensis was filtered out and fermented and aged for more than 6 months. The aged fermented product of pyrus ussuriensis was sterilized at 65° C. for 30 minutes, sealed and stored at room temperature. The prepared fermented product of pyrus ussuriensis was diluted in PBS by 5 times and used.

### Embodiment 2. Production of DSS-Induced Colitis Animal Model

Animals used in the experiment were 8-9 week old male C57BL/6N mice purchased from DBL Co Ltd, Korea. Mice were experimented on after a 7-day adaptation period at the Hongcheon Medical Herb Research Institute animal laboratory, and water and feed were not restricted during the adaptation period. A standardized environment was provided to the experimental animals, day and night were maintained at 12-hour intervals, and room temperature (23±2° C.) and humidity (40-60%) were maintained at appropriate levels. This animal experiment was performed in compliance with all regulations for the management and use of laboratory animals with the approval (approval number: HIMH A20-02) of the Laboratory Animal Control Committee (IACUC) of Hongcheon Medical Herb Research Institute.

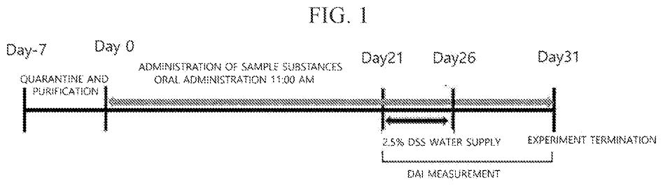

As shown in FIG. 1, colitis was induced by changing the normal drinking water of mice to 25% (w/v) DSS (Dextran Sulfate Sodium salt) for 5 days, and then replaced with purified water to give a recovery period of 5 days. PBS and sample substances were administered into the gastrointestinal tract by inserting a feeding needle catheter (Zonde) into the oral cavity at a dose of 0.2 mL/25 g body weight 3 weeks before the 2.5% DSS treatment. The experimental group was constituted as shown in Table 1 below.

TABLE 1

| Name | Group (n = 8) | Administration substance |
|---|---|---|
| CON | Normal control | PBS |
| DSS | 2.5% DSS | |
| PWE | 2.5% DSS + PWE 400 mg/kg | Hot water extract of pyrus |
| | | ussuriensis |
| PEE | 2.5% DSS + PEE 400 mg/kg | Ethanol extract of pyrus |
| | | ussuriensis |
| PEX | 2.5% DSS + PEX 5 times | Fermented product of pyrus |
| | dilution | ussuriensis |

### Embodiment 3. Analysis of Colitis Treatment Effects of Extract of Pyrus Ussuriensis or Fermented Product of Pyrus Ussuriensis

### 3-1. Evaluation of Diarrhea Index, Bloody Stool Index and Disease Activity Index (DAI)

From the third day after starting the 2.5% DSS water supply, body weight was measured at the same time every morning, and the degree of diarrhea and bloody stool were checked to measure the DAI index.

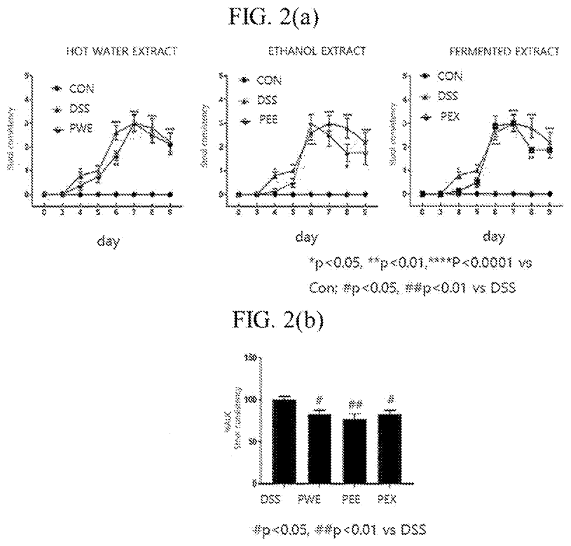

As shown in FIG. 2(a), the degree of diarrhea in the DSS group began to be significantly thinner than that of the control group on the fifth day, and diarrhea began on the sixth day. Diarrhea, a symptom of colitis caused by DSS ingestion, lasted for three days from 6-8 days and then improved. The PWE group had a significantly lower diarrhea index than the DSS group on the fifth day, and the diarrhea index decreased overall. A graph quantifying the diarrhea index is shown in FIG. 2(b). Compared to the DSS group, it was confirmed that % AUC of diarrheal index was significantly decreased in the extract and fermented product of pyrus ussuriensis treated group.

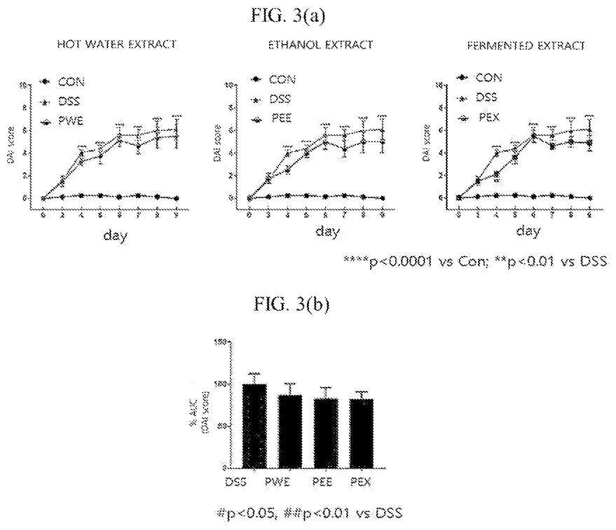

As shown in FIG. 3(a), a lot of bloody stool appeared between 3 and 5 days of ingestion of 2.5% DSS. Upon termination of the DSS water supply, visually confirmed bloody stools decreased significantly, and occult blood in response to the bloody stool analysis kit continued. It was confirmed that the bloody stool index of the group treated with the hot water extract, ethanol extract, and fermented product of pyrus ussuriensis all showed a tendency to decrease overall compared to the DSS group, and specifically, it was confirmed that the bloody stool index decreased significantly in the PEE group and the PEX group on the fourth day compared to the DSS group. A graph quantifying the blood stool index is shown in FIG. 3(b), and it was confirmed that the blood stool index was significantly reduced in the group treated with hot water extract and ethanol extract of pyrus ussuriensis, compared to the DSS group.

Disease activity index (DAI) was measured based on Table 2 below.

TABLE 2

| Score | Weight loss (%) | Stool consistency | Bleeding |
|---|---|---|---|
| 0 | None | Normal | Normal |
| 1 | 0-10% | — | — |
| 2 | 10-15% | Loose stools | Hemocult+ |
| 3 | 15-20% | — | — |
| 4 | >20% | Diarrhea | Gross Bleeding |

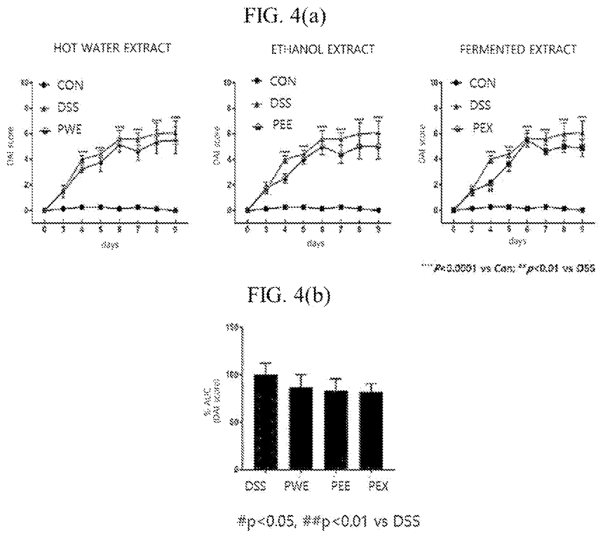

As shown in FIG. 4(a), the DAI score of the PEX group decreased significantly compared to the DSS group on the fourth day of DSS ingestion. A graph quantifying the above DAI score is shown in FIG. 4(b), and it was confirmed that the DAI scores of the PWE, PEE, and PEX groups all decreased compared to the DSS group during the experiment period.

Through this, it was found that the extract and fermented product of pyrus ussuriensis were effective in the treatment of colitis animal models.

### 3-2. Analysis of Inflammatory Cytokines and Inflammatory Response Factors

The proximal colon portion of the animal model was frozen in liquid nitrogen and stored at −80° C. until analysis. The inflammatory cytokine IL-6 was analyzed in plasma, and the concentration of IL-1β was measured in colon tissue.

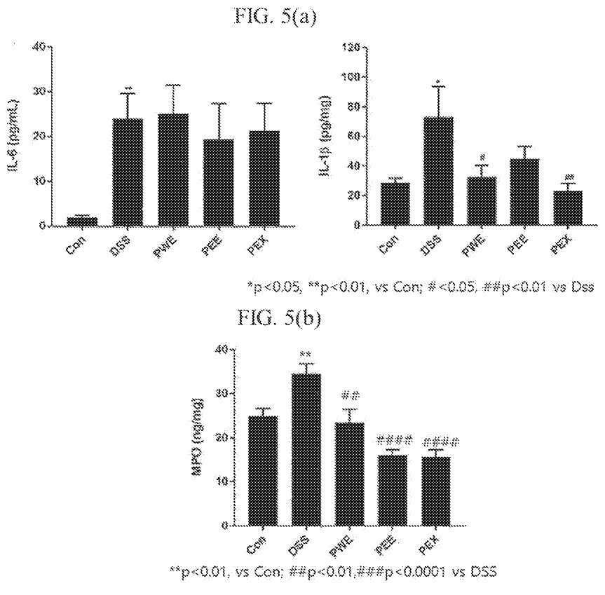

As a result, as shown in FIG. 5(a), it was confirmed that IL-6 in blood increased more than 10 times when treated with DSS compared to the control group, but the concentration of IL-6 decreased in the PEE group. In addition, it was confirmed that the concentration of IL-1β in the colon tissue increased by DSS ingestion decreased in the PWE and PEE groups, and in particular, the concentration of IL-1β was significantly suppressed in the PEX group.

MPO (myeloperoxidase) is myeloid cell-type hydrogen peroxide, and its activity is known to increase when an inflammatory reaction is active. As a result of measuring the concentration of MPO in the colon tissue, as shown in FIG. 5(b), the MPO concentration was greatly increased in the DSS group, but all increased MPO concentrations were significantly suppressed in the PWE, PEE, and PEX groups.

The above results suggest that the extract or fermented product of pyrus ussuriensis is effective in treating inflammation in an animal model of colitis.

From the above description, those skilled in the art to which the present disclosure pertains will be able to understand that the present disclosure can be implemented in other specific forms without changing its technical spirit or essential features. In this regard, it should be understood that the embodiments described above are illustrative in all respects and not limiting. The scope of the present disclosure should be interpreted as including all variations or modifications derived from the meaning and scope of the following claims and equivalent concepts thereof rather than the detailed description above.

What is claimed is:

1. A pharmaceutical composition for prevention or treatment of colitis including an extract of pyrus ussuriensis or a fermented product of pyrus ussuriensis as active ingredient.

2. The pharmaceutical composition according to claim 1, wherein the extract is prepared by extraction with water, alcohol having 1 to 4 carbon atoms or a mixed solvent thereof.

3. The pharmaceutical composition according to claim 1, wherein the fermented product is obtained by mixing and fermenting a pyrus ussuriensis and sugar.

4. The pharmaceutical composition according to claim 3, wherein the sugar is one selected from, sucrose, maltose, glucose, fructose, and refined sugar.

5. The pharmaceutical composition according to claim 3, wherein the fermented product is a mixture of a pyrus ussuriensis and sugar in a weight ratio of 5:5 to 7:3.

6. The pharmaceutical composition according to claim 1, wherein the colitis is selected from a group consisting of ulcerative colitis, inflammatory bowel disease, acute colitis, bacterial colitis, viral colitis, pseudomembranous colitis, chronic colitis, Crohn's disease, ischemic colitis, Behcet's colitis, drug-induced colitis, collagenous colitis, lymphocytic colitis, and radiation colitis.

7. The pharmaceutical composition according to claim 1, wherein the composition provides an effect of reducing disease activity index (DAI), reducing diarrhea index, or reducing bloody stool index.

8. A treatment method for colitis including administering the pharmaceutical composition according to claim 1 to a subject other than humans.

9. A food composition for prevention or improvement of colitis including an extract of pyrus ussuriensis or a fermented product of pyrus ussuriensis as active ingredient.

10. A health functional food composition for prevention or improvement of colitis including an extract of pyrus ussuriensis or a fermented product of pyrus ussuriensis as active ingredient.
